(19) United States
(12) Patent Application Publication
(10) Pub. No.: US 2005/0064587 A1
(43) Pub. Date: Mar. 24, 2005

(54) Pancreatic small cells and uses thereof

(76) Inventors: Lawrence Rosenberg, Quebec (CA); Maria Petropavlovskaia, Montreal (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/489,007

(22) PCT Filed: Sep. 7, 2002
(86) PCT No.: PCT/CA02/01364

(30) Foreign Application Priority Data
Sep. 7, 2001 (US) 60317510

(51) Int. Cl. C12N 5/08

(52) U.S. Cl. 435/366

(57) ABSTRACT

The present invention provides mammalian pancreatic progenitor cells (“small cells”) and methods for their isolation and propagation. The pancreatic small cells are derived from adult pancreatic tissue and are characterised by their small size. The small cells are quiescent or undergo a very slow cell cycle when maintained in cell culture. Small cells secrete synaptophysin and islet hormones and are predominantly found in small, growing islets as small clusters. The present invention further provides for the use of the pancreatic small cells in transplantation and the treatment of diabetes mellitus, and for the genetic engineering of the small cells in order to produce recombinant proteins in vivo.

### FIELD OF THE INVENTION

The present invention pertains to the field of progenitor cells and in particular to progenitor cells from mammalian adult pancreas that have potential to differentiate into mature islet cells.

### BACKGROUND

There has been an increasing interest in recent years in the possible identification of stem or progenitor cells in the pancreas. The availability of such a cell for expansion in vitro and for bioengineering would have a significant therapeutic potential in the treatment of diabetes mellitus [Soria et al. Diabetologia, 44:407-415 (2001)]. Stem and progenitor cells in adults have been isolated primarily from bone marrow and the nervous system and are being extensively studied [Weissman, Cell, 100:157-168 (2000); Fuchs and Segre, Cell, 100:143-155 (2000)]. While it is generally believed that similar cells exist in other tissues and organs as well, actual proof for this is very limited.

Evidence for the existence of a progenitor cell in the pancreas rests primarily on the phenomenon of islet neogenesis that can be experimentally induced by cellophane wrapping of the pancreas [Rosenberg et al., J Surg Res., 35:63-72 (1983)], by partial pancreatectomy [Bonner-Weir et al. Diabetes, 42:1715-1720 (1993)] or by streptozotocin-induced diabetes [Fernandes et al., Endocrinology, 138:1750-1762 (1997)]. Islet neogenesis has also been observed during pregnancy [reviewed in Bonner-Weir, Endocrinology, 141:1926-1929 (2000)]. Although a protein that induces islet neogenesis (Islet Neogenesis Associated Protein, INGAP) has been purified and characterized and its cDNA cloned [Rosenberg, Microscopy Research and Technique, 43:337-346 (1998); Rafaeloff et al., J. Clin. Invest., 99:2100-2109 (1997)], a potential target-progenitor cell remains unknown.

The pancreatic duct is currently favoured as a potential source of progenitor cells in adult pancreas [Rosenberg, Microscopy Research and Technique. 43:337-346 (1998); Bonner-Weir, PNAS, 97:7999-8004 (2000)] since development of islets during embryogenesis is known to be closely associated with ductal epithelium [Madsen et al., Eur. J. Biochem., 242:435-445 (1996]. The presence of scattered endocrine cells within the adult ductal system has also been reported [Gu and Sarvetnick, Development, 118:33-46 (1993)], as has budding of new islets from the small ducts [Bouwens and Pipeleers, Diabetologia, 41:629-633 (1998)]. A recent report which indicated that ductal cells can expand in vitro and form insulin-producing islet-like structures further supports the pancreatic duct as the source of pancreatic progenitor cells [Bonner-Weir, et al., PNAS, 97:7999-8004 (2000); Ramiya et al., Nature Medicine, 6:278-282 (2000)].

Recently, an analysis of islet regeneration in the mouse pancreas after administration of streptozotocin has suggested that beta progenitor cells, which differentiate into insulin-producing cells following injury, may be present in the islets [Guz et al., Endocrinology, 142:4956-4968 (2001)]. This study proposed two putative progenitor cells, one which expresses Glut-2 and another which co-expresses insulin and somatostatin. Another report has demonstrated that rat and human islets contain a distinct population of nestin-positive and hormone-negative immature cells, which proliferate extensively in vitro and appear to be multipotential [Zulewski et al., Diabetes, 50:523-533 (2001)]. Whether these cells participate in islet regeneration and neogenesis in vivo has yet to be determined.

In contrast to the putative pancreatic progenitor cells described above, hematopoietic stem cells and hepatic oval cells remain dormant and undifferentiated most of the time and resume proliferation only when a need for regeneration arises. A pancreatic progenitor cell that similarly remains dormant but has the potential to differentiate into a functioning beta-cell when necessary would have widespread applications in the treatment of diabetes.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide pancreatic small cells and uses thereof.

In accordance with an aspect of the invention there is provided a population of adult pancreatic cells comprising pancreatic progenitor cells that have a diameter of about 15μ or less and which express synaptophysin and PDX-1.

In accordance with another aspect of the invention there is provided a method for isolating a population of adult pancreatic cells comprising pancreatic progenitor cells comprising:
(a) providing a preparation of adult pancreatic islets;
(b) culturing said islets under standard cell culture conditions in a cell culture medium to form a cell monolayer;
(c) maintaining said monolayer by replenishment of said cell culture medium whereupon pancreatic progenitor cells can be identified therein by their small size of less than about 15μ in diameter, and
(d) isolating said small cells from the monolayer.

In accordance with yet another aspect of the invention there is provided a method of genetically engineering a population of pancreatic progenitor cells that have a diameter of about 15μ or less and which express synaptophysin and PDX-1, to introduce a gene capable of expressing a recombinant protein, said method comprising:
(a) providing a gene encoding said protein, and
(b) introducing said gene into one or more cells in said population.

In accordance with a further aspect of the invention there is provided a method of immortalizing pancreatic progenitor cells comprising the step of transfecting said cells with a suitable immortalizing gene.

In accordance with another aspect of the invention there is provided a method of transforming, transfecting or transducing a population of pancreatic progenitor cells that have a diameter of about 15μ or less and which express synaptophysin and PDX-1, to introduce a polynucleotide operatively associated with a heterologous regulatory sequence that controls or alters the gene expression of said cells.

In accordance with yet a further aspect of the invention there is provided a use of pancreatic progenitor cells to generate insulin-secreting cells for transplantation into a mammal.

In accordance with yet a further aspect of the invention there is provided a use of pancreatic progenitor cells to generate pancreatic islets for transplantation into a mammal.

Methods for the treatment of diabetus, or other disease conditions of the pancreas, such as pancreatic cancer, are also provided for, including cell or tissue replacement therapies to complement primary cancer therapies.

### BRIEF DESCRIPTION OF THE FIGURES

FIG. 1 provides a schematic representation of potential pathways to pancreatic beta-cells.

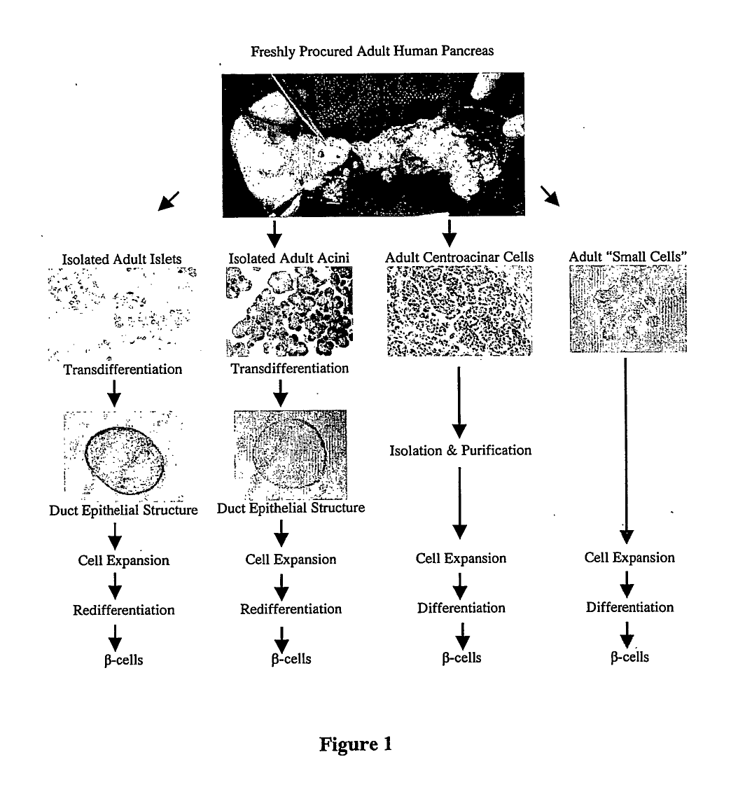

FIG. 2 demonstrates the in vivo model for islet cell neogenesis and islet formation. (A) depicts pancreatic duct to beta-cell differentiation in a Syrian hamster treated with a novel growth factor called INGAP peptide (insulin staining in red). (B) depicts insulin-positive islet-like structures forming amidst acinar tissue in a Syrian hamster pancreas (insulin staining in red). (C) depicts new beta-cells forming amidst acinar tissue in an INGAP-peptide treated dog (insulin staining in brown).

FIG. 3 demonstrates that pancreatic small cells are highly quiescent and depicts a number of their characteristics. These cells are characterized by their extremely small size (approximately 5-10 μm in diameter), their appearance singly or in small clusters (A), their rather immature ultrastructural appearance with poorly developed rER and Golgi apparatus, few mitochondria and the presence or absence of immature endosecretory granules (B), and on immunocytochemical screening, the presence of markers such as alpha-fetoprotein (C), Bcl-2 (D), and synaptophysin (E).

FIG. 4 depicts a sub-population of pancreatic small cells that synthesises and stores insulin and that responds to a glucose challenge by actively secreting insulin. (A) depicts insulin expression in the small cells (human) cultured for 3 weeks. Insulin is in red. (B) provides an example of glucose-stimulated insulin release by small cells cultured for 3 days after isolation.

FIG. 5 demonstrates that overlaying pancreatic small cells in culture with type 1 collagen (an extracellular matrix component) augments the ability of these cells to release insulin into the culture medium.

FIG. 6 depicts a phase-contrast micrograph of a 2-month old primary culture of canine islets showing two groups of small cells clearly visible among large cells. The three-dimensional clusters of small cells (arrows) are typical for these cultures. Bar is 10A.

FIG. 7 presents electron micrographs of a small cluster of human pancreatic cells obtained by sieving islets through a 25μ pore-size mesh. (A) arrows indicate insulin granules containing a crystal core, “Mu” indicates mucin droplets, white arrows indicate glucagon granules and arrowheads indicate finger-like membrane invaginations. (B) depicts a fragment of a small cell co-expressing insulin (arrows) and somatostatin (arrowheads). Bars are 1μ.

FIG. 8 depicts the results of immunocytochemical staining of primary islet cultures. Small cells are immunopositive for: (A) synaptophysin (human cells, 1 week after plating); (B) insulin (canine cells, 1 week); (C) glucagon (canine cells, 1 week); (D) somatostatin (human cells, 2 weeks); (E) PDX-1 (human cells, 6 weeks); (F) nestin (human cells, 1 week-small cells are negative, insert shows a nestin-positive cell from the same culture); (G) alpha-fetaprotein (canine cells, 1 week); (H) Bcl-2 (human cells, 4 weeks). Small cells are surrounded by other islet cells, which stain negative for all these markers. Bars are 10μ.

FIG. 9(A) depicts islet cell monolayers that were continuously labelled with BrdU for 4 weeks. Arrowheads indicate areas of BrdU-negative small cells amongst a few positive “big” cells; (B) depicts islets that were embedded into Matrigel™ and cultured with BrdU for two weeks, recovered with dispase and allowed to spread on plastic without BrdU. Arrows indicate BrdU positive cells, arrowheads indicate BrdU-negative cells. Cells were slightly counterstained with hematoxylin. Bar is 10μ

FIG. 10 depicts insulin release by human small cells (A) and islets (B) in response to glucose challenge. Insulin content in the culture media is expressed as mean±S.E.M.

FIG. 11 depicts a cluster of tightly packed small cells in a monolayer of rat islet cells which has been labelled with BrdU. Note that three of the small cells have BrdU positive nuclei.

FIG. 12 depicts (A) a small cluster of a 3-4 pancreatic small cells on epithelial monolayer without collagen overlay. (B) depicts the same culture after 10 days of type 1 collagen overlay.

### DETAILED DESCRIPTION OF THE INVENTION

Definitions

Unless defined otherwise, all technical and scientific terms used herein have the same meaning as commonly understood by one of ordinary skill in the art to which this invention pertains.

The term “progenitor cell” as used herein refers to an undifferentiated pancreatic cell that has the potential to differentiate into a mature α-, β- or γ-cell. The term progenitor cell thus encompasses cells that are multipotential, such as stem cells, and cells that are pre-committed to differentiating into cells of a specific lineage.

The term “cluster” as used herein with reference to pancreatic small cells means a group of at least two associated cells. Typically, clusters are groups of about six to eight cells, but the clusters may comprise as many as about 50 cells. The cells within the cluster are tightly packed and within the cluster at least two of the cells are linked by a desmosomal structure.

The term “quiescent” as used herein with reference to pancreatic small cells means that the cell is currently not undergoing repeated cell cycles, but may be stimulated to do so.

The term “cell cycle” as used herein refers to the sequence of events between mitotic cell divisions, which lead up to and cumulate in the division of the cell into two daughter cells.

Pancreatic Progenitor Cells

The present invention provides a population of small, undifferentiated, quiescent or slow cycling pancreatic cells (“small cells”) that can be isolated from adult mammalian pancreas. These pancreatic small cells are progenitor cells which have the potential to differentiate into mature islet cells. The small cells may be multipotential or they may be pre-committed to differentiate into a certain type of mature islet cell, i.e. an α-, β- or γ-cell. In accordance with the present invention, the small cells belong to the neuroendocrine lineage and have an immature phenotype. Pancreatic small cells can be isolated, for example, from primary cultures of pancreatic islets and typically represent 1% or less of the total islet cell population.

1. Characteristics of Pancreatic Small Cells

The pancreatic small cells of the present invention can be characterised primarily by their small size. Thus, the small cells have a diameter of about 15μ or less. Typically, the small cells have a diameter of between about 5μ and about 15μ. In one embodiment of the present invention, the small cells have a diameter of between about 5μ and about 12μ. In a related embodiment, the small cells have a diameter of between about 7μ and about 10μ.

The pancreatic small cells of the present invention are either quiescent or undergo a slow cell cycle. When maintained under standard cell culture conditions, the small cells retain their small size and immature phenotype. In one embodiment of the present invention, the small cells remain unchanged under standard cell culture conditions for up to four months.

Pancreatic small cells, when cultured, may occur singly or in small clusters of tightly packed cells. When present in clusters, finger-like invaginations and/or desmosomes may be present between two or more of the cells. Clusters of small cells are typically difficult to separate by standard techniques, such as enzymatic treatment and/or treatment with EDTA. For example, standard protease and/or dispase treatment is generally insufficient to separate the cells within a cluster. In one embodiment of the present invention, the small cells remain clustered after treatment with trypsin for about seven minutes.

In accordance with the present invention, the pancreatic small cells belong to the neuroendocrine lineage and, therefore, express and are capable of reacting with antibodies to synaptophysin. In contrast to mature cells, the pancreatic small cells continue to express synaptophysin for prolonged periods in culture. In one embodiment of the present invention, the small cells express synaptophysin for at least one week in culture. In a related embodiment, the small cells express synaptophysin for at least four weeks in culture.

Pancreatic small cells also express and react with antibodies to the stem cell markers α-fetaprotein and Bcl-2. In addition, the small cells may express and react with antibodies to one or more of the proteins: glucagon, insulin, somatostatin, polypeptide P, LIF receptor, TGFβ Receptors type I and II and PDX-1. An isolated population of pancreatic small cells in accordance with the present invention may comprise several sub-populations, which express different combinations of the above proteins.

In one embodiment of the present invention, a sub-population of the small cells expresses insulin. Pancreatic small cells that express insulin typically do so in a glucose-dependent manner. Thus, addition of high concentrations (for example, 22 mM) of glucose to the culture medium can stimulate secretion of insulin by the small cells therein. In a related embodiment of the present invention, glucose-dependent secretion of insulin by the pancreatic small cells is not enhanced by addition of known secretagogues, such as arginine or 3-isobutyl-1-methylxanthine (IBMX) to the culture medium, in contrast to mature islets. Thus, in this embodiment, the mechanisms regulating the insulin release in the pancreatic small cells is different from mature β-cells. In contrast to mature β-cells in culture, small cells continue to secrete insulin for prolonged periods. In one embodiment of the present invention, the small cells that secrete insulin continue to do so for up to 10 weeks in culture.

Expression of other genes and/or proteins by the small cells can be determined by standard techniques in the art. For example, expression profiling is a rapid and convenient method of determining which genes in a cell are being expressed and the level of expression of each gene relative to an appropriate control [see, for example, the review by Buchholz et al., Pancreatology, 1:581-586 (2001)]. This approach can be used to identify other “marker” genes or proteins for the small cells, by using mature islet cells as controls to which gene expression in the small cells is to be compared.

Typically, expression profiling makes use of pre-fabricated microarrays of short DNA sequences or oligonucleotides. Microarrays comprise an ordered arrangement of thousands of oligonucleotides, each capable of specifically hybridrising to a certain gene, immobilised onto a suitable solid support. Suitable supports for microarrays include, for example, nitrocellulose, plastic, nylon and glass. Typically microarrays useful for this purpose represent between 1,000 and 40,000 genes. Methods of constructing microarrays are well known in the art [see, for example, Ausubel, et al., Current Protocols in Molecular Biology, John Wiley & Sons, Inc, NY. (1989 and updates)]. In addition, custom-made microarrays are available from many companies. Pre-made microarrays are also commercially available for many organisms including, for example, GeneChip® (Affimetrix, Santa Clara, Calif.), Atlas™ (BD Biosciences-CLONTECH, Palo Alto, Calif.), GEM Microarrays, GeneJet™ array and LifeSeq® (Incyte Genomics, Palo Alto, Calif.), MICROMAX™ Human cDNA Microarray Systems (PerkinElmer Life Sciences, Boston, Mass.) and ResGen™ GeneFilters®, (Invitrogen, Huntsville, Ala.).

For expression analysis, RNA is isolated from the small cells and from control cells. If necessary, the RNA can be amplified by conventional techniques to ensure a sufficient quantity for analysis. The RNA is than hybridised to the microarray under suitable conditions and a routine analysis of the microarray by commercially available scanners and software is conducted to identify genes whose expression is altered in the small cells relative to the control cells. Suitable hybridization conditions can readily be determined by one skilled in the art using standard techniques.

In one embodiment of the present invention, expression profiling is used to identify levels of gene expression relative to mature islet cells. In a related embodiment, higher expression levels of the genes encoding SIAT1 (sialyltransferase 1), ASCL1 (achate-scute complex homologue, transcriptional factor), CECR1 (cat eye syndrome chromosome region related-adenosine deaminase) and SV2, are identified in human small cells cultured for about two weeks when compared with freshly isolated islets.

2. Isolation and Propagation of Pancreatic Small Cells

The pancreatic small cells of the present invention are isolated from adult mammalian pancreas. Islets are first isolated from the pancreas by standard techniques known in the art, such as enzymatic digestion and/or mechanical dissociation [see, for example, Ricordi et al., Diabetes, 37:413-420 (1988); Paraskevas et al., FEBS Lett., 455:203-208 (1999); Wang & Rosenberg, J. Endocrin., 163:181-190 (1999)]. Blends of enzymes suitable for this purpose are also commercially available (for example, Liberase® Enzyme Blends from Roche Diagnostics; Serva Collagenase from Crescent Chemical). In one embodiment of the present invention, islets are isolated from canine or human pancreata by digestion with Liberase® followed by semi-automated dissociation and purification using EuroFicoll.

Small cells can be subsequently isolated from cultures of the islet preparations. The islet preparations are cultured directly in an appropriate medium using standard cell culture techniques such that a monolayer of cells is formed within the culture vessel. The monolayer cultures are maintained by replenishment of the medium at appropriate time intervals.

Alternatively, the islet preparation can be passed through an appropriately sized sieve prior to culture, for example, a sieve with a mesh size of less than 140μ. In one embodiment of the present invention, the islets are passed through a sieve with a mesh size between about 25μ and about 140μ. Clusters of pancreatic small cells pass intact through the sieves with mesh sizes of 25μ or more, therefore, sieving the islets prior to culture results in a preparation that is substantially enriched in small cells. In addition, small islets, for example those that pass through a 100μ mesh, contain a higher proportion of small cells than larger islets. The sieved preparation can then be cultured as described above.

Small cells can be distinguished amongst the other cells in the monolayer by their small size and their tendency to appear in small rounded clusters. With prolonged culture times, the small cells become more obvious. For example, after about two months in culture, the morphology of the small cells remains substantially the same while that of the surrounding cells changes. Pancreatic small cells, either individually or in small clusters, can be isolated from the monolayers using, for example, a cloning cylinder or by handpicking with a pipette tip. The small cells can be further identified by their expression of one or more phenotypic markers, such as α-fetoprotein and Bcl-2, or by the unique expression profile of one or more genes or proteins, such as SIAT1, ASCL1, CECR1 or SV2. Expression of phenotypic markers by the small cells can be readily determined by standard techniques, such as immunocytochemistry techniques. Levels of expression of genes or proteins can be determined using standard expression profiling techniques.

The small cells can also be isolated based on their size using techniques known in the art, for example, counterflow elutriation or density gradient centrifugation.

Alternatively, the pancreatic small cells can be isolated based on the expression of one or more phenotypic markers of these cells such as, Bcl-2 or α-fetoprotein, or other markers identified by expression profiling. For example, the small cells can be isolated by contacting an islet cell suspension with one or more monoclonal antibodies specific for Bcl-2 or α-fetoprotein. Cells bound by the antibody can then be separated and recovered. The antibodies can be coupled to a solid support according to known techniques in order to facilitate recovery of the cell:antibody complex. For example, the antibody may be coupled to a magnetic bead or to a biotin or streptavidin molecule. Alternatively, the small cells can be isolated by fluorescence activated cell sorting (FACS) techniques based on the expression of, for example, Bcl-2 or α-fetoprotein.

The isolated small cells can be transferred into a culture vessel with or without subsequent trypsinization and/or treatment with EDTA or dispase and cultured under standard conditions. Alternatively, when pancreatic small cells are prepared from a monolayer, they can be left in culture with the large epithelial cells of the monolayer, which act as a feeder layer and facilitate long-term culture.

The pancreatic small cells of the present invention are either quiescent or have a very slow growth cycle. Typically the cell cycle of the pancreatic small cells in vitro is greater than or equal to two weeks. Thus the small cells can be maintained long term either in standard culture or on an extracellular matrix. In accordance with the present invention, the small cells do not substantially alter their morphology in long-term culture and can be maintained in culture for up to at least four months.

For long-term storage, the small cells of the present invention can be frozen using standard cryopreservation techniques.

3. Proliferation of Pancreatic Small Cells Ex Vivo

In accordance with the present invention, the pancreatic small cells either do not proliferate under standard cell culture techniques, or they do so only very slowly. For example, canine and human pancreatic small cells do not-proliferate under standard culture conditions in the absence or presence of one of the mitogens EGF, FGF-2, HGF, SCF, glucose (up to 22 mM), GLP-1, nicotinamide, LIF or INGAP, or a combination thereof, nor do they proliferate when grown on extracellular matrices such as collagen, laminin, gelatine, fibronectin or Matrigel™. In contrast, rat pancreatic small cells proliferate very slowly under standard culture conditions with a cell cycle of approximately two weeks. The ability of the small cells to proliferate can be readily determined by techniques known in the art, for example, by monitoring the uptake of bromodeoxyuridine (BrdU) by the cells.

Proliferation of pancreatic small cells, however, may be stimulated by maintenance of the three dimensional structure of the islet. This can be achieved, for example, by embedding small islets in a suitable matrix, such as collagen or Matrigel™ using methods known in the art [for example, see Yuan et al., Differentiation, 61:67-75 (1996)].

Genetically Engineered Pancreatic Small Cells

The pancreatic small cells described herein, and their differentiated progeny can be immortalised or conditionally immortalised using standard techniques known in the art. For example, the cells may be transfected with a suitable immortalising gene, including, but not limited to, simian virus 40 large T antigen, papillomaviruses E6 and E7, adenovirus E1A, Epstein-Barr virus, human T-cell leukaemia virus, herpesvirus saimiri, oncogenes, and mutant p53 gene [see, for example, Katakura et al., Methods Cell Biol., 57:69-91 (1998)]. Alternatively, the small cells can be conditionally immortalised using techniques such as Tet-conditional or reversible immortalization (see, for example, WO 96/31242; Efrat et al, 1995, PNAS, 92,3576-3580, describing transfection with SV-40 T antigen (Tag) under control of the tetracycline (tet) operon), or Mx-1 conditional immortalization (see, for example, WO 96/02646).

The pancreatic small cells of the present invention can be genetically engineered using known techniques such that they are capable of expressing recombinant proteins in vitro or in vivo. Methods of genetically engineering mammalian cells are well-known in the art [see, for example, Ausubel et al., Current Protocols in Molecular Biology, John Wiley & Sons, New York (1989 and updates)]. Typically, the gene encoding the protein of interest is inserted into a suitable expression vector which is subsequently introduced into the small cells in vitro. The gene may be operably linked to a regulatory control sequence that aids expression of the encoded protein. The gene may also be fused to a heterologous sequence which encodes another protein or fragment thereof, such that a fusion protein is expressed. Alternatively a vector may be used to introduce a regulatory sequence into the small cells in order to regulate expression of a target gene (see, for example, U.S. Pat. Nos. 5,641,670 and 5,733,761). The regulatory sequence integrates into the chromosome of the cell at a site upstream of the target gene and exerts an activating or repressing effect on the expression of the gene in the homologously recombinant cell. As such, it is possible to activate a gene which is normally not expressed, to increase the expression of a gene which is not expressed at physiologically significant levels, to change the pattern of regulation or induction of a gene, or to reduce (including eliminating) a gene's expression in small cells as obtained.

Methods or introducing genes and/or vectors into cells are known in the art and include, for example, standard transfection, transduction and transformation techniques, such as calcium phosphate co-precipitation, DEAE-dextran transfection, lipofection, electroporation, microinjection, biolistics, or viral transfection. Mammalian transfection kits are also available commercially (for example, from Stratagene, La Jolla, Calif.; Invitrogen, Carlsbad, Calif.).

Use of the Pancreatic Small Cells

The present invention contemplates the use of the pancreatic small cells for a variety of applications, including drug screening, genomics and transplantation. For example, in vitro cultures of small cells can be used to detect and evaluate growth factors or inhibitory factors important in the differentiation of pancreatic progenitor cells into committed lineages, or to produce pancreatic tissue suitable for assessment of the effects and/or toxicity of various pharmaceutical compounds. For example, the cells can be used to assess the effects of glucose sensitisers in enhancing insulin synthesis and secretion; mitotic and anti-mitotic agents; apoptosis and anti-apoptosis agents, and chemotherapeutics.

The pancreatic small cells can be used in genomics studies and applications, for example, to develop a better understanding of the developmental biology of adult pancreatic progenitor cells and thus provide new information into signal transduction mechanisms and with respect to receptors and small molecules that could be used for guiding cell lineage programming. Genomics approaches may also provide additional insight in to pancreatic carcinogenesis.

Pancreatic small cells, clusters or differentiated progeny thereof can be transplanted into a: patient in order to treat a variety of pancreatic diseases and disorders, for example states of endocrine deficiency such as diabetes, or to replace damaged, lost or diseased pancreatic tissue. Alternatively, the transplanted cells may augment the function of the endogenous host tissue. For example, pancreatic small cells that secrete insulin and are capable of differentiating into mature β-cells, or the differentiated progeny thereof, can be used for transplantation into a patient suffering from a condition characterized by a deficiency of insulin, such as one of the various forms of diabetes mellitus. Alternatively, pancreatic small cells can be administered to a cancer patient who has undergone chemotherapy to kill cancerous pancreatic cells in order to replace the damaged pancreatic tissue.

The pancreatic small cells for transplantation purposes may be derived from the patient (i.e. autologous) or they may be derived from a heterologous source. If the pancreatic small cells are derived from a heterologous source, concomitant immunosuppressive therapy is typically administered as is known in the art.

The pancreatic small cells, or differentiated progeny thereof, can be administered to a mammal in need thereof by conventional techniques such as direct transplantation or by injection or infusion. Injection or infusion can be local, i.e. directly into the pancreas, or systemic and can be achieved using standard delivery devices provided that the needle lumen or bore is of sufficient diameter (for example, 30 gauge or larger) to prevent shear forces damaging the small cells. The injectable small cell preparations can also be administered intravenously, either by continuous drip or as a bolus.

The cells may be transplanted or infused alone or in association with a pharmaceutically acceptable carrier or medium. The present invention contemplates the use of the carrier or medium to introduce other compounds, such as immuno-suppressive compounds, therapeutic compounds mitogenic compounds or differentiating agents, into the patient in conjunction with the small cells.

Alternatively, the cells can be embedded in a biocompatible medium such as an extracellular matrix that will promote survival and/or proliferation and differentiation of these cells in vivo. The matrix can function as a “scaffolding” that holds the small cells in place at the site of injury. Or the pancreatic small cells can be administered in a biocompatible medium becomes a semi-solid or solid matrix in situ. Such extracellular matrices are known in the art and may be a natural matrix or may be a matrix that is based on natural polymers, such as collagen and its derivatives, fibronectin, polylactic acid or polyglycolic acid (for example, Gelfoam from Upjohn, Kalamazoo, Mich.). Alternatively, the matrix may be based on bio-synthetic polymers [see, for example, Jeong, et al., Adv. Drug Deliv. Rev., 54:37-51 (2002); U.S. Pat. Nos. 6,388,047 and 6,384,105]. The present invention also contemplates the incorporation of other therapeutically useful compounds into the matrix with the cells such that the cells and the compound can be delivered concomitantly to the pancreas, such as a compound which exerts a therapeutic effect in the mammal or that produces a biologically active molecule that has a growth or trophic effect on the transplanted cells, or that induces differentiation of the pancreatic small cells into a particular phenotypic lineage.

Genetically engineered pancreatic small cells can be administered to a patient in order to produce a therapeutically effective recombinant protein in situ. Typically, the small cells are isolated from a donor, transfected or transformed with a recombinant gene in vitro and transplanted into the patient whereupon the recombinant protein is expressed. Methods of genetically engineering cells are known in the art and include those described herein and elsewhere. The genetically engineered cells can be transplanted or administered to the patient as described above. The use of genetically engineered pancreatic small cells can thus serve the dual purpose of replacing or augmenting a patient's pancreatic tissue and delivering a therapeutic protein in situ.

Kits

The present invention also contemplates kits comprising the pancreatic small cells for research or therapeutic purposes.

The small cells are provided in the kit in a suitable form for long-term storage, for example, the small cells may be frozen and the kit subsequently maintained at an appropriate temperature (for example, −20° C. or −70° C.). The kit may further comprise suitable media and reagents for culturing the cells and/or inducing proliferation of the cells. The kit may also comprise a suitable extracellular matrix for use with the cells for transplantation purposes. Individual components of the kit may be packaged in separate containers. The kit may further comprise instructions for use.

For therapeutic purposes, the kit may also comprise one or more instruments for assisting with the injection or placement of the cells within the body of a mammal (such as a syringe, pipette, forceps, eye dropper or similar medically approved delivery vehicle). The kit may further comprise a notice in the form prescribed by a governmental agency regulating the manufacture, use or sale of biological products, which notice reflects approval by the agency of the manufacture, use or sale for human or animal administration.

To gain a better understanding of the invention described herein, the following examples are set forth. It should be understood that these examples are for illustrative purposes only. Therefore, they should not limit the scope of this invention in any way.

### EXAMPLES

### Example 1

### In Vivo Model for Islet Cell Neogenesis

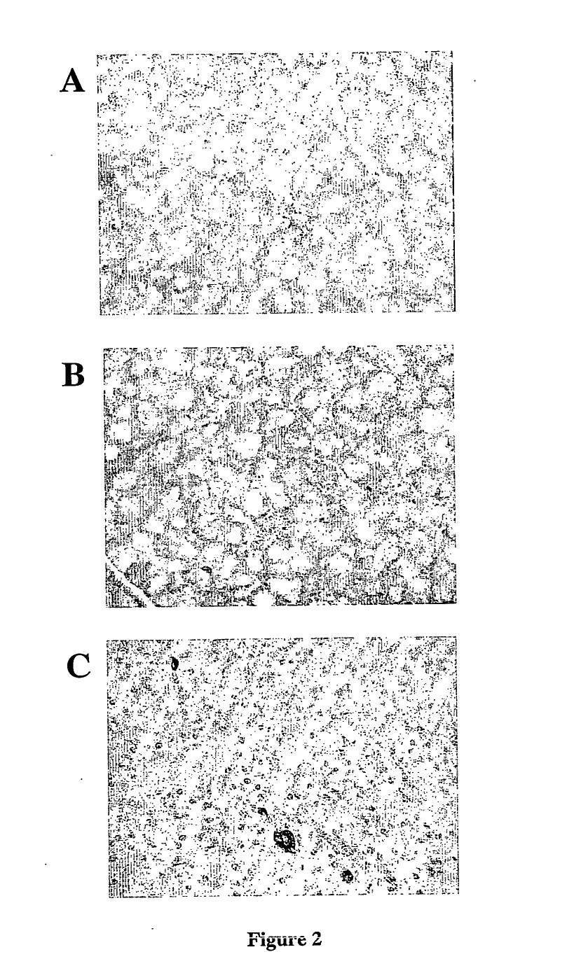

An in vivo model for islet cell neogenesis and islet formation has been developed [Rosenberg et al., J. Surg. Res, 35:63-72 (1983)], in which new islets appeared to be derived from cells associated with the ductal epithelium (FIG. 2A).

In the model, islet cell neogenesis is mediated by the novel acinar cell protein INGAP [Rafaeloff et al., JCI, 99:2100-2109 (1997)]. While many of the new islets clearly bud from adjacent ductal epithelium, a careful re-analysis of the tissues from over one thousand hamster pancreata, and more recently a study of canine pancreata exposed to INGAP peptide, have demonstrated that new β-cells also appear (and small islets form) amidst the exocrine tissue (FIG. 2B, C), independent of duct epithelium.

The appearance of these new β-cells indicates that previously unrecognised progenitor cells are present in the pancreas, which give rise to the new β-cells. While immature cells have been shown to be scattered diffusely throughout the parenchyma of the pancreas, unassociated with ductal epithelium [Bouwens et al., Diabetologia, 41:629-633 (1998)], these cells have remained poorly characterized. The present invention describes the identification of pancreatic progenitor cells and provides methods to isolate and purify them.

### Example 2

### Preliminary Isolation, Purification and Characterisation of Pancreatic Small Cells

Small cells were first identified in cellular digests of cadaveric human pancreata that had been processed for islet isolation for transplantation. Human islet isolation was performed by the semi-automated method originally proposed by Ricordi [Ricordi, et. al., Diabetes, 37:413-420 (1988)]. Procured pancreases are distended by intra-ductal infusion of Liberase® HI (Roche Molecular Biochemicals, Indianopolis, Ind.) or Serva Collagenase (Cresent Chemical, Brooklyn, N.Y.) [Linetsky et al., Diabetes, 46:1120-1123 (1997)], and then dissociated using the automated method [Ricordi, et. al, Diabetes, 37:413-420 (1988)]. The separation occurs during a process of continuous digestion lasting approximately 12-30 minutes, after which the digestion circuit was cooled and the tissue collected into approximately 8 litres of cold Hanks solution and washed. Liberated islets were separated from non-islet tissue on a continuous gradient of Euroficoll in a Cobe 2991 cell separator.

Preparations of partially purified islets from the Cobe cell separator were then passed through a series of different size steel mesh screens (100 to 25μ pores), and the retrieved tissue was placed into culture directly on plastic in CMRL with 10% FCS and permitted to spread out. In the process, the cells of the islet de-differentiated and rapidly proliferated to produce a monolayer that contained primarily large epithelial-like cells and fibroblastoid cells. Pancreatic small cells were identified in these cultures. To isolate the small cells from the monolayers, either cloning cylinders or handpicking with a pipette tip was used. The cells were then transferred into a new plate with or without subsequent trypsinization or/and treatment with EDTA or dispase. Alternatively, small cells were left in culture with the large epithelial cells of the monolayer as a feeder layer that appeared to facilitate long-term culture.

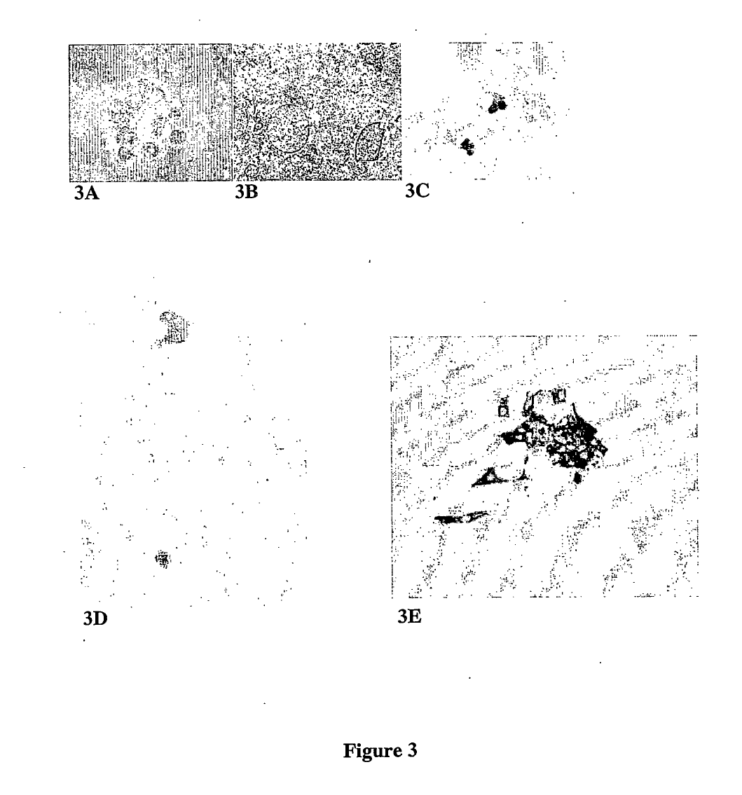

Pancreatic small cells are quiescent and are characterized by their small size (approximately 5-10 μm in diameter), their appearance singly or in small clusters (FIG. 3A), their rather immature ultrastructural appearance with poorly developed rER and Golgi apparatus, few mitochondria and the presence or absence of immature endosecretory granules (FIG. 3B), and on immunocytochemical screening, the presence of markers such as, alpha-fetoprotein (FIG. 3C), Bcl-2 (FIG. 3D), and synaptophysin (FIG. 3E) that are consistent with a stem cell, perhaps of neuroendocrine lineage, but in a state of primitive or intermediate differentiation.

The small cells were either organised into small clusters, which can be identified within islets, or were independent of islets. The appearance of small cells in islets was inversely related to islet size, such that few small cells were located in larger islets (>250 μm), and many such cells were localised in smaller islets (<100 μm). Small cells appear to represent less then 1% of islet cells.

Besides being identified within islets, small cells can be isolated and purified from a fraction of small tissue clusters that pass through a 25 μm pore size mesh.

Small cells are quiescent in culture and can be maintained in culture for at least up to four months. They do not incorporate BrdU (bromo-deoxyuridine, a measure of DNA synthesis and hence proliferation) unlike the rapidly growing cells of the underlying epithelial cell monolayer.

Another feature of the small cells was that they are very difficult to dissociate from each other by trypsinization such that they remained clustered together even after 7 minutes in trypsin, while longer trypsinization impaired their viability.

Insulin Production by Pancreatic Small Cells

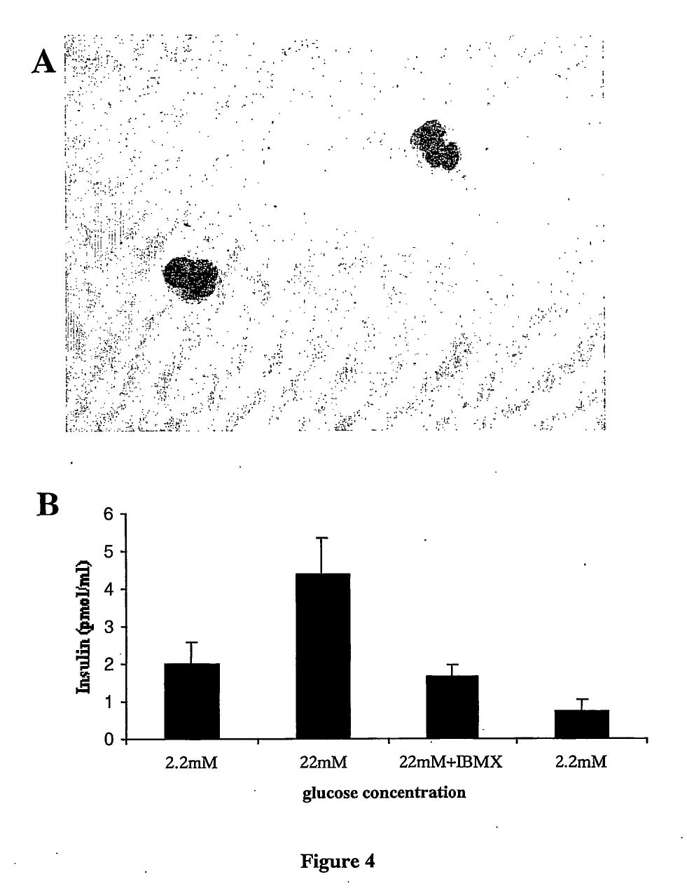

A sub-population of the small cells synthesized and stored insulin (FIG. 4A) and responded to a glucose challenge by actively secreting insulin (FIG. 4B). Unlike true pancreatic endocrine cells which lose their insulin expression almost immediately after spreading out from islets cultured on plastic, this sub-population of small cells continued to secrete insulin for at least up to 10 weeks in culture.

Effect of Extracellular Matrix (ECM) on Insulin Production and Growth

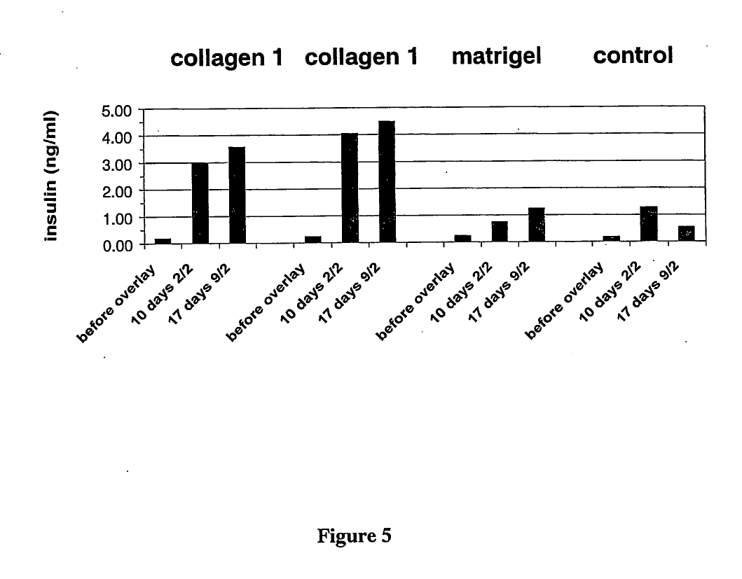

Overlaying small cells in culture with an extracellular matrix such as type 1 collagen augmented the ability of these cells to release insulin into the culture medium. Matrigel™ had much less effect on insulin release (see FIG. 5).

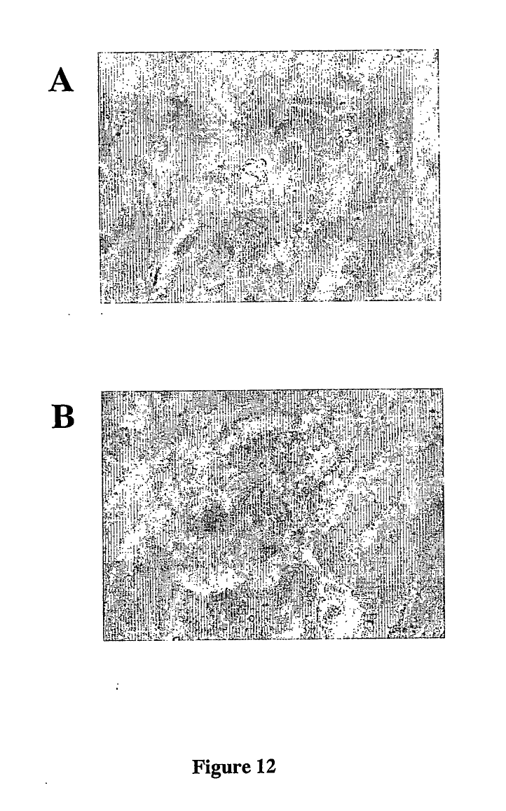

When small cells in CMRL with 10% FCS were maintained on an islet-derived epithelial monolayer and then overlaid with type 1 collagen, they will grow (see FIG. 12). It is possible that the increase in insulin release shown in FIG. 5 was due in part to small cell proliferation as well as an increased production and release of insulin.

### Example 3

### Isolation and Propagation of Canine and Human Pancreatic Small Cells

Pancreata from mongrel dogs (2-4 years old with body weight 20-25 kg) were removed under general anaesthesia in accordance with Canadian Council for Animal Care (CCAC) guidelines. Human pancreata were obtained from heart-beating cadaveric donors following in situ flush with University of Wisconsin solution at the time of multi-organ harvest for transplantation. Prior consent for organ donation was obtained by the local procurement organisation Quebec-Transplant. Cold ischemia time varied between 4 to 8 hours.

Islet isolation from both canine and human pancreas was carried out using enzymatic digestion with Liberase® CI and Liberase® HI (Roche Diagnostics, Laval, Que., Canada) for canine and human pancreas respectively, mixed with 0.1 mg/ml DNase I (Roche Diagnostics, Laval, Que., Canada). Digestion was followed by semi-automated dissociation and EuroFicoll purification as previously described [Paraskevas et al, FEBS Lett., 455:203-208 (1999); Wang and Rosenberg, J. Endocrin., 163:181-190 (1999)]. The final islet preparations were 70-90% dithizone-positive. Islets were resuspended in the regular culture medium CMRL-1066 (GIBCO BRL, Burlington, Ont., Canada) supplemented with 10% FBS (Montreal Biotech, Montreal, Que., Canada), penicillin, streptomycin and fungizone (GIBCO BRL, Burlington, Ont., Canada) and counted under an inverted microscope.

Aliquots of islets (1000 islets/10 ml) were transferred into 10 cm tissue culture plates and allowed to adhere to plastic for 48-72 hours. The cultures were maintained for different time intervals (up to 4 months) with media change every 3 days. In a number of experiments freshly isolated islets were sieved through stainless steel meshes (Bellco Biotechnology, Vineland, N.J., USA) with pore size 25, 94 and 140 microns to fractionate islets and cell clusters by size.

Sieved islets were cultured either on plastic or on extracellular matrices such as Collagen type I, either rat tail [prepared according to Richards et al, J. Tissue Cult. Methods, 8:31-36 (1983)] or bovine (gift from Dr. S. Sullivan, Organogenesis, Canton, Mass.); pig gelatin 0.1% (Sigma-Aldrich, Oakville, Ont., Canada); human fibronectin 5 μg/cm²; mouse laminin 5 μg/cm² and Matrigel™, thin gel method (all from Becton-Dickinson, Mississauga, Ont., Canada), prepared according to the manufacturers suggestions. In a number of experiments islets were embedded into collagen, as described [Yuan et al., Differentiation, 61:67-75 (1996)] or Matrigel™ (thick gel method).

Freshly isolated islets, plated into tissue culture plates, immediately attach to the plastic. Within a couple of days cells start migrating from the islets, forming rounded areas of polygonal and elongated cells. The dynamics of cell attachment and spreading are basically the same for canine and human islets. In both species this cell population appears to be quite heterogeneous at first but becomes more homogenous as cells form a monolayer after approximately a week in culture. As cells spread out of the islet core, they usually increase in size and lose their endocrine phenotype.

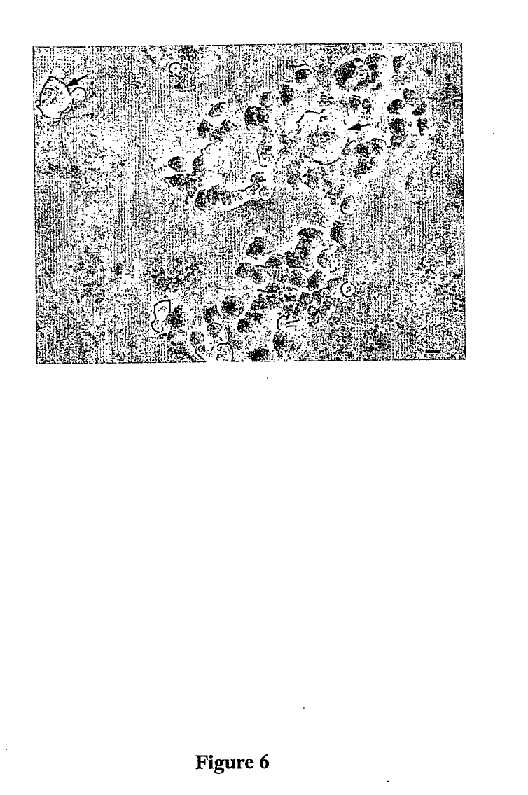

Among the variety of cell types forming a monolayer, scattered groups of small cells (about 7-10μ) were found, which were either flattened or clustered into small rounded clusters that were very distinct from the other cells (FIG. 6). Single cells of this type can also be seen. These cells were scarce and represented less than 1% of total islet cell population. Distinctive features of these cells became more obvious with prolonged periods in culture—unlike most cells, they remained small, rounded or hexagonal in shape and usually maintained close cell contacts with each other (FIG. 6).

### Example 4

### Characterisation of Pancreatic Small Cells by Electron Microscopy and Immunocytochemistry

Processing of samples for electron microscopy was performed in the Department of Pathology, Montreal General Hospital. Cell clusters were fixed in 1.0% glutaraldehyde/4% formaldehyde in 0.1M phosphate buffer, pH 7.4, at 4° C., for 30 min or longer, and postfixed in 1.0% osmium tetroxide in 0.1M phosphate buffer for 30 min at 4° C. Following dehydration through a graded series of acetons, samples were embedded into Epon 812 and polymerized at 60° C. overnight. Ultrathin sections 60 nm were cut on Ultracut-5 ultramicrotome, stained with uranyl acetate and lead citrate and examined on a Philips CM10 electron microscope.

Immunostaining was performed for insulin, glucagon, somatostatin (BD PharMingen, Mississauga, Ont., Canada) (dilution 1:100), CK-19 (1:50), alpha-fetaprotein (1:100), c-kit (1:100), CD34 (1:50), Bcl-2 (1:80), pancreatic polypeptide (1:500), synaptophysin (1:100) (Dako Diagnostics, Mississauga, Ont., Canada), nestin (1:200) (Chemicon International, Temecula, Calif., USA), LIF receptor and TGFO Receptors type I and II (Santa-Cruz Biotechnology, Santa Cruz, Calif., USA, 1:200). Antibody for PDX-1 was a kind gift from Dr. C. Wright, Vanderbilt University, Nashville, Tenn., USA. The Insulin, c-kit, CD34 and Bcl-2 antibodies were monoclonal mouse anti-human IgG1 type, while the rest of the antibodies were rabbit anti-human.

Monolayers were fixed with either 4% paraformaldehyde at 4° C., 10% formalin at room temperature or methanol at −20° C., 10 min, depending on the suggestions of the antibody manufacturers. Plates were stained with multiple primary antibodies using greased 8 mm cloning cylinders (Bellco Biotechnology, Vineland, N.J., USA), which were attached to the areas of interest after plates were washed with PBS and blocked with 5% lamb serum (GIBCO BRL, Burlington, Ont., Canada). Primary antibodies (50-100 μl per cylinder) in appropriate dilutions were added for overnight incubation at 4° C. Normal rabbit serum and IgG1 isotype antibodies (same dilution) were used as negative control to exclude nonspecific staining. After washing several times with PBS, antigens were detected using biotin-HRP based Histostain-plus bulk kit and AEC (red) chromogen/substrate system (Zymed Laboratories, San Francisco, Calif., USA). Alternatively, ABC detection kit (Vector Laboratories, Burlington, Ont., Canada) was used, followed by DAB staining.

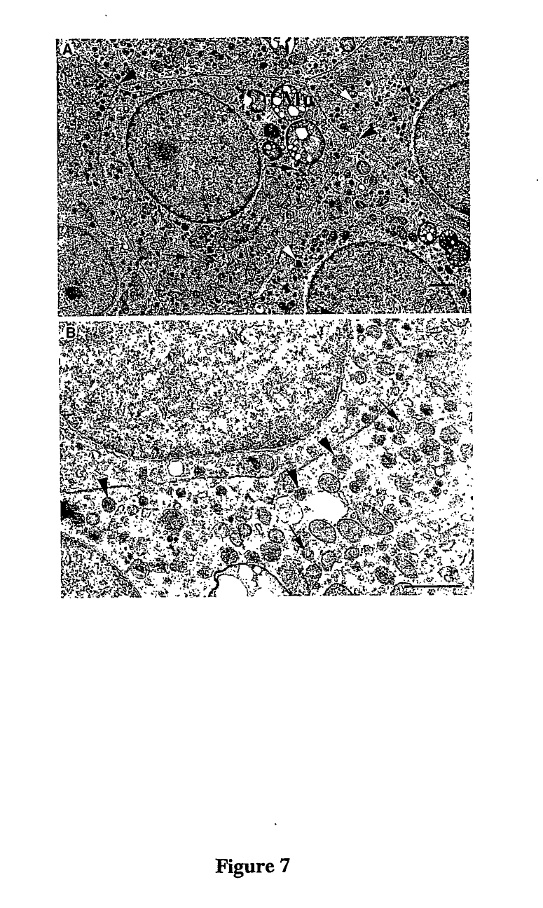

Ultrastructurally, the appearance of small cells was consistent with a fairly undifferentiated phenotype—the cytoplasm contained poorly developed endoplasmic reticulum and Golgi complex, several small mitochondria and a small number of neuroendocrine granules (FIG. 7A). The neuroendocrine granules were of different types, including typical insulin, glucagon and somatostatin granules as well as undefined types of granules. Typically, one cell contained predominantly one type of granules but sometimes granules of different type can be observed in the same cell. For example, the cell in the center of FIG. 7A contains only typical insulin granules characterized by a dense, often crystal-like core (black arrows). Two adjacent cells on the right contain predominantly glucagon-like dense granules without a core (white arrows) but a few insulin granules as well. Coexistence of somatostatin and insulin granules in the same cell was also observed (FIG. 7B). Some cells also contained a few mucin droplets, but exhibited no other ductal markers. A distinctive feature of small cells grouped in clusters was that they were tightly packed together with finger-like invaginations between cell membranes (FIG. 7A, arrowheads) and desmosomes. This fact is consistent with the observation that cells in the small clusters are very difficult, sometimes impossible to dissociate by enzymatic treatment and/or EDTA.

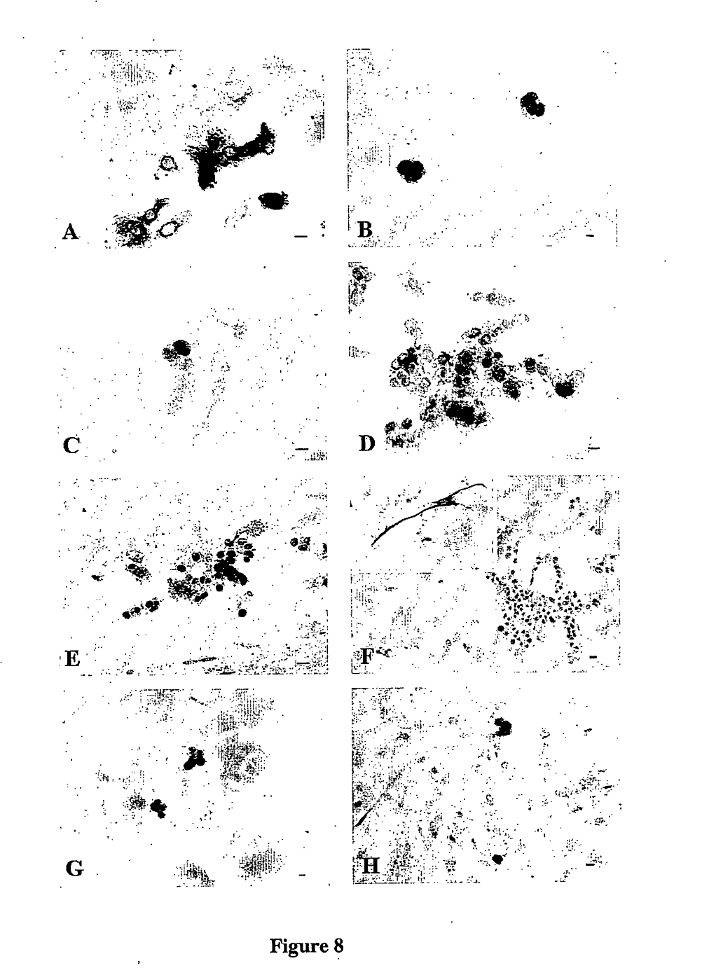

Results of immunostaining, summarized in Table 1 (below) and FIG. 8, were consistent overall with the electron microscopic observations, indicating that small cells belong to the neuroendocrine lineage. Thus, synaptophysin—a protein characteristic for neurosecretory granules, appears to be the most reliable marker for small cells (FIG. 8A). Small cells maintained in tissue culture plates were positive for insulin; glucagon, somatostatin (FIG. 8B-D) and polypeptide P (Table 1). It should be noted that small cells maintained expression of synaptophysin and the islet hormones for a few weeks, whereas the rest of the cell monolayer, represented by dedifferentiated islet cells of a large size, lost expression of these compounds within a few days (FIG. 8A-D).

Although electron microscopy indicated that some small cells contained more than one type of secretory granule, no double immunopositive cells were detected (data not shown). This fact suggests that despite an immature phenotype, small cells are committed to different lineages. The possibility, however, that immunostaining is not sensitive enough to detect a small amount of a co-expressed marker cannot be excluded. Importantly, most of the small cells expressed Pdx-1, a transcription factor required for insulin gene transcription and associated with pancreatic development and regeneration. The typical nuclear localization of Pdx-1 was visible in small cells but not in the underlying cell monolayer (FIG. 8E). Although some small cells contained mucin droplets (as indicated above), they did not react with antibodies to cytokeratin 19, a marker of ductal phenotype. Small cells did not immunoreact with antibodies for such stem cell markers as CD34, c-kit and nestin (Table 1, FIG. 8F), but stained positive for alpha-fetaprotein and Bcl-2 (FIG. 8G, H).

To assess the distribution of small cells among the islets, freshly isolated islet preparations were fractionated using a number of screens with different pore size. Small cells were found within most of the islets but their frequency appeared to inversely correlate with the islet size—they were predominantly found in small islets (diameter<150 μm), while big islets (>300 μm) contained very few, if any, such cells. Besides being found within islets, small cells can be collated from in a fraction of small clusters that passes through a 25 μm pore size mesh. These clusters were present in all canine and human islet isolation preparations examined, and appeared to be independent structures rather than chipped islet fragments produced by over-digestion during islet isolation. The small cells in clusters had the same morphology as cells found in the islets.

TABLE 1
IMMUNOPHENOTYPING OF SMALL CELLS

| Marker | Staining |
|---|---|
| Insulin | + |
| Glucagon | + |
| Somatostatin | + |
| Polypeptide P | + |
| Synaptophysin | + |
| α-fetaprotein | + |
| Bcl-2 | + |
| Pdx-1 | + |
| LIFR | + |
| TGFβ1 RI, RII | + |
| TGFβ1, 2, 3 | − |
| Vimentin | − |
| Nestin | − |
| CK 19 | − |
| c-kit | − |
| CD34 | − |
| Thy-1 | − |
| Amylase | − |

### Example 5

### BrdU Immunostaining of Pancreatic Small Cells

To assess DNA replication in the canine and human small cells 0.1 mM BrdU (Sigma-Aldrich, Oakville, Ont., Canada) was added to the culture medium for 24 h, 72 h, 7 days, 14 days and 28 days. In the case of long-term labelling, the medium was exchanged every two days for fresh medium with freshly added BrdU. Incorporated BrdU was detected with monoclonal anti-BrdU antibody, purchased from Sigma-Aldrich or Amersham Pharmacia Biotech (Baie d'Urfe, Quebec, Canada) followed by Histostain-plus/AEC system (Zymed Laboratories, San Francisco, Calif., USA) as described in Example 4.

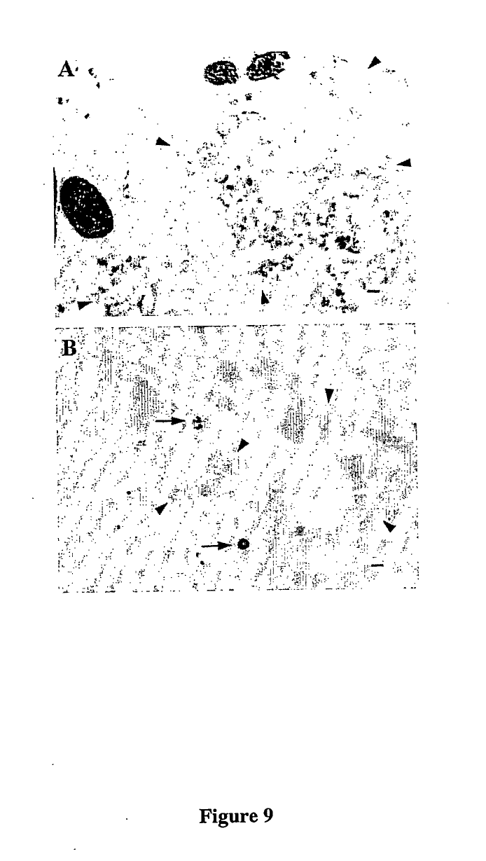

In tissue culture canine and human small cells appear to be extremely quiescent such that groups of these cells remain virtually unchanged for up to four months, when maintained in the same flask with regular media changes. Remarkably, when the cultures were exposed to continuous labelling with BrdU for up to four weeks, small cells failed to incorporate the label while the rest of the cells in the same culture plates (dedifferentiated islet cells) incorporated BrdU at a rate of almost 100%, as expected for such a prolonged labelling period (FIG. 9A). Quiescence of small cells can present a real challenge for their characterisation in tissue culture. Because of their quiescence, low numbers and small size they are easily lost in mixed cultures when monolayers are subcultured and other, rapidly proliferating cells take over. To isolate small cells from monolayers cloning cylinders were used or the cells were hand-picked with a pipette tip and transferred into a new plate with or without subsequent trypsinization and/or treatment with EDTA or dispase.

Attempts to stimulate proliferation of the small cells using a number of factors, such as EGF, FGF-2, HGF, SCF, glucose (up to 22 mM), GLP-1, nicotinamide, LIF and INGAP, used alone or in combination, and with and without serum were not successful. Similarly, a number of extracellular matrices, including type 1 collagen, laminin, fibronectin and Matrigel™ had no apparent effect on canine and human small cell quiescence. It should be noted that the lack of proliferation does not seem to depend on the cell density. Thus, subculturing of small cells at different densities, both alone and in combination with other cells from monolayers, did not prove to be successful in stimulating proliferation.

Since small cells appear to prefer to stay clustered, it is possible that they depend on signaling mediated by direct cell-cell contacts and 3-D islet structure, which is disrupted when the islets spread. To test whether small cells are able to proliferate in the intact islets, small islets (40-80μ) were embedded into collagen (type I) or Matrigel™ to prevent spreading and to preserve the islet structure, and were cultured for 2 weeks in the presence of BrdU. After 2 weeks, the cultures were treated with collagenase or dispase respectively, the collected islets were washed and allowed to spread on plastic for a week or two without BrdU in the medium, followed by fixation and immunostaining with a BrdU-specific antibody. Remarkably, as shown in FIG. 9B, single BrdU-positive small cells, usually one or two cells per cluster, were found, although the frequency of the clusters containing positive cells was very low. This indicates that small cells do proliferate within the islets but they appear to be very slow cycling cells with a cell cycle longer than two weeks. This also suggests that direct contacts with other islet cells may be important for small cell proliferation.

### Example 6

### Insulin Secretion by Pancreatic Small Cells

Glucose-responsiveness of cultured cells was studied in the course of glucose challenge experiments, carried out in RPMI supplemented with 0.5% BSA and containing glucose in either low (2.2 mM) or high (22 mM) concentration as previously described [Wang and Rosenberg, J. Endocrin., 163:181-190 (1999)]. Approximately 5000 human small cells were plated into a 6-well plate immediately after islet isolation, and 48 hours later, were subjected to a course of glucose stimulation. Handpicked human islets (50) were maintained in non-tissue culture plates for 48 hours after isolation and then underwent the same course of glucose stimulation.

The course of glucose stimulation was as follows: Low glucose 60 min−High glucose 30 min−High glucose+50 μM 3-isobutyl-1-methylxanthine (IBMX)/or 20 mM arginine (Sigma) for 30 min−Low glucose 60 min.

To measure the basal secretion of insulin, samples of culture media were harvested 24 hours after media change and collected at −20° C. until further testing. Quantitative determination of insulin was performed using 1-2-3 Porcine Insulin ELISA kit and 1-2-3 Human Insulin ELISA kit (ALPCO Diagnostics, Windham, N.H., USA) for canine and human samples respectively.

To determine if small cells secreted insulin, the culture medium was tested at different time intervals after islet isolation, using the Insulin ELISA kit. For these experiments small human clusters, less than 25 μm in diameter, were used. These clusters were either sieved as described above, or handpicked and cultured in 6 well plates in CMRL 1066, containing 101% FBS and 5.5 mM glucose. The number of dithizone positive cells was counted after the samples of medium were taken. Roughly, small cells secrete 3 fmols/cell/24 hours if tested within 72 hours after islet isolation.

To determine whether small cells were glucose-responsive, insulin secretion was studied in the course of glucose challenge experiments carried out on small clusters of cells 48-72 hours after isolation in RPMI supplemented with 0.5% BSA and containing glucose in either low (2.2 mM) or high (22 mM) concentration.

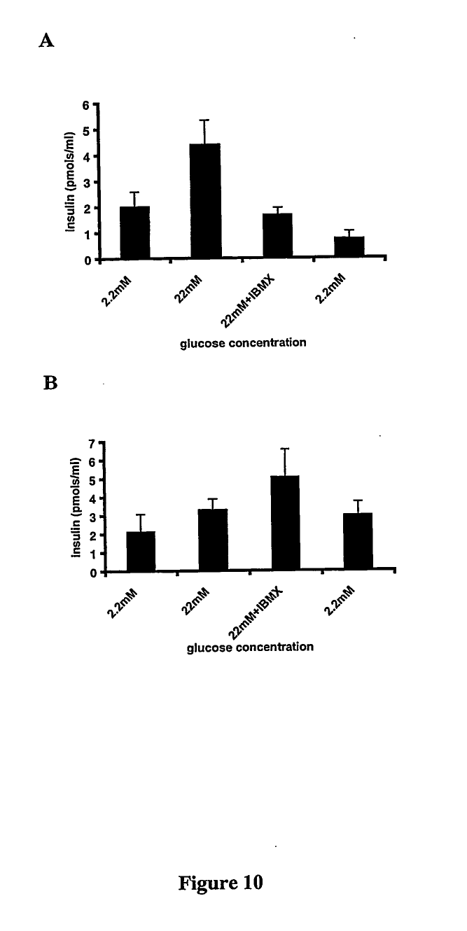

The data from 4 experiments, summarized in FIG. 10A, show that insulin production by small clusters was glucose responsive—there was a 2.2 fold increase in insulin concentration after 30 min exposure to 22 mM glucose. Notably, this increase could not be further enhanced by either IBMX or another secretagogue, 20 mM arginine (data not shown). In contrast, IBMX and arginine seemed to inhibit the insulin secretion compared to high glucose alone. Human islets used as a control in this experiment responded to IBMX with higher or the same insulin release compared to high glucose alone (FIG. 10B). These data demonstrate the glucose responsiveness of small cells but suggest that mechanisms regulating the insulin release in small cells might be different from mature beta cells.

Unlike mature beta cells that lose insulin expression immediately after spreading out of islets, the small cells continue secreting insulin for up to 8-10 weeks in culture, although in gradually decreasing amounts. This provides another indication that the small cells represent a distinctive cell population within pancreatic islets.

### Example 7

### Small Cells Isolated From Rat Pancreas

Small cells were also isolated from rat pancreatic islets following a similar protocol as that described above for canine and human small cells. In brief, rat islets were isolated from pancreata of normal Wistar rats by digestion with collagenase IV followed by purification on a BSA or Ficoll gradient. The islets were allowed to spread on TC plates for 2 weeks. 50 ug/ml BrdU was added continuously for 2 more weeks, followed by fixation in methanol (15 min at −20° C.) and immunodetection of BrdU.

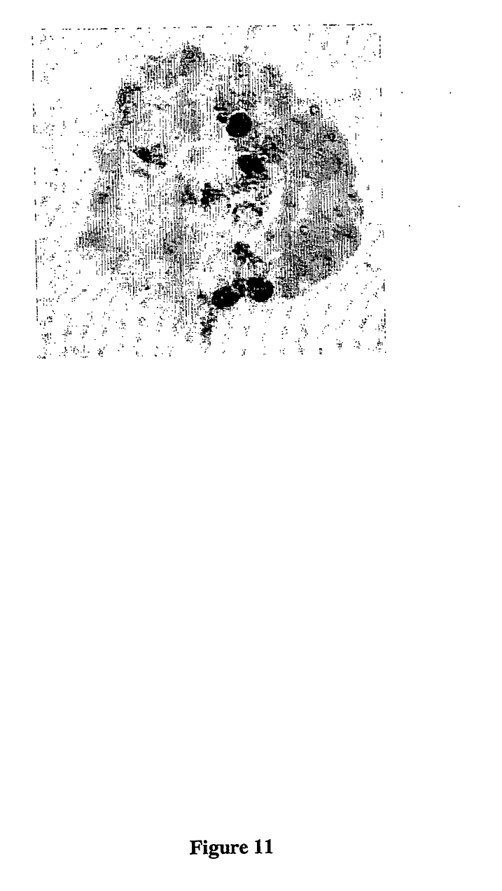

Rat small cells have the same morphology as human and canine pancreatic small cells and are organized in similar types of clusters as can be seen in FIG. 11, which shows a cluster of tightly packed small cells. Note that three of the small cells have BrdU positive nuclei. Thus, unlike their human counterparts, rat small cells in the spread islet monolayers incorporate BrdU, albeit at a low rate. The approximate length of the cell cycle of the rat pancreatic small cells is two weeks, which is similar to that of human small cells when embedded in an extracellular matrix as described above.

These data indicate that: a) small cells are capable of proliferation; b) human and rat small cells require different regulatory signals, and c) rat small cells are a convenient model to study the potential of small cells as putative progenitors.

### Example 8

### Preliminary Expression Profiling of Human Pancreatic Small Cells

Preliminary data derived from a study on genomic profiling, indicate that human small cells cultured for about two weeks express much higher (>10×) levels of certain genes when compared with freshly isolated islets. These include, among others, SIAT1 (sialyltransferase 1), ASCL1 (achate-scute complex homologue, transcriptional factor), CECR1 (cat eye syndrome chromosome region related-adenosine deaminase) and SV2, as well as some currently uncharacterised genes. Human islets were allowed to spread in tissue culture plates for one to two weeks until (pancreatic) small cells became well visible, then clusters of small cells were picked with a pipette tip under an inverted microscope and collected for subsequent RNA extraction. 50-100 ng of total RNA from 200-300 small clusters (about 3-5000 cells) were extracted and the RNA then amplified for analysis using a commercially available kit (Arcturus, Calif.). To determine which genes are up- or downregulated in the mall cells, RNA from small cells was compared with RNA from intact, freshly isolated islets, which contain predominantly mature endocrine cells. RNA from both sources was treated identically, including the amplification procedure. The micro-array analysis was performed at the Montreal Genome Center using Affymetrix GeneChips®.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A population of adult pancreatic cells comprising pancreatic progenitor cells that have a diameter of about 15μ or less and which express synaptophysin and Pdx1.

2. The population of adult pancreatic cells according to of claim 1, wherein said pancreatic progenitor cells have a diameter of between about 5, and about 12μ.

3. The population of adult pancreatic cells of claim 1, wherein said pancreatic progenitor cells further express one or more proteins selected from the group consisting of glucagon, insulin, somatostatin, polypeptide P, LIF receptor, TGFβ Receptors type I and II, α-fetoprotein and Bcl-2.

4. The population of adult pancreatic cells of claim 3, wherein a subpopulation of said pancreatic progenitor cells express insulin.

5. The population of adult pancreatic cells of claim 4, wherein said subpopulation of said pancreatic progenitor cells express insulin in a glucose-dependent manner.

6. The population of adult pancreatic cells of claim 1, wherein said pancreatic progenitor cells are quiescent in standard cell culture.

7. The population of adult pancreatic cells of claim 1, wherein said pancreatic progenitor cells have a cell cycle of at least about 2 weeks in standard cell culture.

8. A method for isolating a population of adult pancreatic cells comprising pancreatic progenitor cells comprising:
(a) providing a preparation of adult pancreatic islets;
(b culturing said islets under standard cell culture conditions in a cell culture medium to form a cell monolayer;
(c) maintaining said monolayer by replenishment of said cell culture medium whereupon pancreatic progenitor cells can be identified therein by their small size of less than about 15μ in diameter, and
(d) isolating said small cells from the monolayer.

9. The method of claim 8, wherein said preparation of adult pancreatic islets is passed through a sieve with a mesh size of less than about 140μ prior to step (b).

10. The method of claim 8, wherein said pancreatic progenitor cells occur usually in clusters comprising at least two progenitor cells but also as single cells.

11. The method of claim 8, wherein said progenitor cells can be further identified by their expression of synaptophysin and PDX1.

12. (cancelled)

13. A method of genetically engineering a population of pancreatic progenitor cells that have a diameter of about 15μ or less and which express synaptophysin and PDX-1, to introduce a polynucleotide capable of expressing a recombinant protein, comprising:
(a) providing polynucleotide capable of expressing a recombinant said protein, and
(b) introducing the polynucleotide into one or more cells in said population.

14. The method of claim 13, wherein the polynucleotide is inserted into an expression vector prior to step (b).

15. The method of claim 13, further comprising the step of transfecting said progenitor cells with a suitable immortalizing gene.

16. The method of claim 15, wherein said progenitor cells are conditionally immortalized.

17. The method of claim 12, wherein the polynucleotide further comprises a heterologous regulatory sequence that controls the expression of said recombinant protein.

18-22. (cancelled)

23. The method of claim 12, wherein the population of cells is engineered to produce insulin secreting cells.

24. The method of claim 12, wherein the population of cells is engineered to produce pancreatic islet cells.

25. The method of claim 12, wherein the population of cells is further prepared for transplantation.

26. The method of claim 25, wherein the population of cells is prepared for transplantation into a human subject.
